(19) United States
(12) Patent Application Publication
(10) Pub. No.: US 2026/0298839 A1
(43) Pub. Date: Oct. 1, 2026

(54) SURFACE DEFECT DETECTION SYSTEM

(71) Applicant: AGTEKS ORME VE TEKSTIL ENDUSTRILERI SANAYI VE TICARET LIMITED SIRKET, Istanbul (TR)

(72) Inventor: Mehmet AGRIKLI, Istanbul (TR)

(21) Appl. No.: 19/489,927

(22) PCT Filed: Jun. 4, 2024
(86) PCT No.: PCT/TR2024/050594

§ 371 Date: Dec. 4, 2025

(30) Foreign Application Priority Data
Jun. 7, 2023 (TR) 2023/006658

(51) Int. Cl. G01N 21/898; G01N 21/88; G01N 21/89; G06T 7/00; H04N 23/55; H04N 23/56; H04N 23/695

(52) U.S. Cl. CPC G01N 21/8983; G01N 21/8851; G01N 21/8901; G06T 7/0004; H04N 23/55; H04N 23/56; H04N 23/695; G06T 2207/20021; G06T 2207/30124

(57) ABSTRACT

The present invention provides a system for actively detecting defects on the surface of an object with a surface area. The system comprises a calibratable image capturing unit configured to capture images from at least one target area of the object, the image capturing unit comprising an image capturing device and optionally a first mirror configured to transmit images from at least one target area to the image capturing device; an image sensor converting the images taken by the image capturing unit into data; a processor communicating with the image sensor to analyze the data received from the image sensor; and at least one light source, characterized in that at least the image capturing device or the optional first mirror is configured to perform a rotational oscillation.

### TECHNICAL FIELD

The present invention relates to a system that actively enables optical detection of defects on the surface of objects.

### BACKGROUND OF THE INVENTION

Some objects are manufactured through continuous processes. For instance, objects like textile products can be produced continuously by flat or circular weaving or knitting machines. Circular manufacturing machines contain a rotating component. A uniform pattern is desired on the surface of such objects. For various reasons, defects can occur in the surface patterns of the objects, and a uniform pattern may not be achieved. For example, in a circular or flat knitting machine, defects in the surface pattern of the final textile product may occur due to reasons such as one of the knitting needles breaking or failing to catch the yarn to be included in the knitting, or the yarn taken from the bobbin (to be included in the knitting) not being of the desired standard quality.

In machines that continuously produce textile products, such as knitting or weaving machines, it is important to detect defects on the product surface quickly. As the textile product progresses longitudinally while being knitted, it is desirable to quickly detect the defect and terminate the production when a defect occurs in the textile product, as the uniformity of the product will be compromised.

Detecting defects on the surface of objects like textile products with the human eye is not always easy, and using human resources for such detection is not preferred in terms of production efficiency. Therefore, optical-based solutions have been particularly developed for defect detection. For example, WO 2021/156756 describes a defect detection system that can be adapted to a circular knitting machine producing tubular textile products. WO 2021/156756 essentially describes an optical defect detection system comprising an imaging unit with a camera that captures images at short intervals and a sensor producing data corresponding to the captured images; a processor interfaced with the sensor for analyzing sensor data, and optionally, an illumination unit.

In the system disclosed in WO 2021/156756, one or more cameras can be used and these cameras can be positioned in various parts of the circular knitting machine. According to a first configuration of WO 2021/156756 (e.g., FIG. 6), multiple cameras are arranged side by side to capture an area close to the top of the winding roll where the knitted textile product is wrapped. In this configuration, the cameras are connected to the rotating component of the knitting machine and thus rotate along with the knitted textile material. In other words, the images captured by the cameras consist of views taken in the direction of the knitted textile product.

According to a second configuration of WO 2021/156756 (e.g., FIG. 9B), a camera is arranged to capture the area where the knitted tubular textile material takes on a shape similar to an inverted triangular pyramid. In this configuration, the camera may or may not be connected to the rotating component of the knitting machine. When the camera is not connected to the rotating component, meaning it is stationary, the images captured by this camera essentially consist of views around the circumference of the knitted textile product. When the camera is connected to the rotating component, the images captured by this camera cannot enable precise defect detection across the entire width of the textile product.

According to a third configuration of WO 2021/156756, similar to the first configuration (e.g., FIGS. 11A to 11C), multiple cameras arranged side by side can be collectively moved horizontally and vertically.

WO 2021/156756 contains several disadvantages. Firstly, it is not possible to detect defects at every stage of the knitted textile product with the same camera configuration. This is because tubular textile products take on different shapes at different stages of production: initially, just below the knitting needles, they take a shape similar to an inverted cone; later, when the textile product reaches the part with separator arms, it takes on a shape similar to an inverted triangular pyramid, and finally, it is passed through tension rollers to be formed into a sheet. In configurations using a single camera (e.g., FIGS. 10A and 10B), it is not feasible to use a high-speed and high-resolution camera, which is very expensive, in the sections where the textile product is brought into sheet form. (e.g., FIG. 6), as the images captured by a single camera cannot enable precise defect detection across the entire width of the textile product. On the other hand, the necessity of using multiple cameras in the parts where the textile product is formed into a sheet increases costs and results in a structure that is not compact, thus complicating the adaptation to a circular knitting machine. A similar disadvantage also applies to the illumination unit.

A further disadvantage of WO 2021/156756 relates to the image capture speed. For instance, in a configuration using a single camera (e.g., FIG. 9A), when the camera is in a fixed position, the image capture speed of the camera is limited by the speed of the rotating component of the circular knitting machine. On the other hand, when the camera is connected to the rotating component, it is not possible for this single camera to capture the entire width of the textile product precisely. Additionally, even if multiple cameras arranged side by side (e.g., FIG. 6) are moved collectively in horizontal and vertical directions (e.g., FIGS. 11A to 11C), this movement cannot reach high speeds, as the cameras are connected to the rotating component of the machine and can be adversely affected by the centrifugal force on the camera and its components.

### BRIEF DESCRIPTION OF THE INVENTION

The object of the invention is to provide an effective detection of defects on the surfaces of objects.

Another object of the invention is to provide a compact system for detecting defects on the surfaces of objects.

A further object of the invention is to ensure the rapid detection of defects on the surfaces of objects.

To achieve these objectives, the present invention provides a system for actively detecting defects on the surface of an object with a surface area. The system comprises a calibratable image capturing unit configured to capture images from at least one target area of the object, the image capturing unit comprising an image capturing device and optionally a first mirror configured to transmit images from at least one target area to the image capturing device; an image sensor converting the images taken by the image capturing unit into data; a processor communicating with the image sensor to analyze the data received from the image sensor; and at least one light source, characterized in that at least the image capturing device or the optional first mirror is configured to perform a rotational oscillation.

According to some exemplary embodiments of the invention, in a configuration where the image capturing unit does not include a first mirror, the image capturing device may perform rotational oscillation preferably via a controlled motor such as a step motor or a servo motor. According to some exemplary embodiments of the invention, in a configuration where the image capturing unit comprises a first mirror, the image capturing device may be held stationary, and the first mirror may perform rotational oscillation preferably via a controlled motor such as a step motor or a servo motor. According to some exemplary embodiments of the invention, in another configuration where the image capturing unit comprises a first mirror, both the image capturing device and the first mirror may perform rotational oscillation preferably via controlled motors such as step motors or servo motors.

According to some exemplary embodiments of the invention, a first light source may be configured to perform rotational oscillation, preferably via a controlled motor such as a step motor or a servo motor, to direct light to the target area of the object. According to some exemplary embodiments of the invention, a second or a third mirror configured to reflect light from the first light source onto the object may be provided. According to some exemplary embodiments of the invention, the second and the third mirror may be configured to perform rotational oscillation, preferably via a controlled motor such as a step motor or a servo motor. According to some exemplary embodiments of the invention, the first light source and the second and the third mirror may be configured to perform rotational oscillation, preferably via controlled motors such as step motors or servo motors.

According to some exemplary embodiments of the invention, a second light source may be provided. This second light source may also perform rotational oscillation, preferably via a controlled motor such as a step motor or a servo motor, similar to the first light source. Similarly, a third mirror configured to reflect light from the second light source onto the object may be provided. According to some exemplary configurations of the invention, the third mirror may be configured to perform rotational oscillation, preferably via a controlled motor such as a step motor or a servo motor. In such a configuration, the image capturing device may be arranged to be located between the first light source and the second light source, preferably at an equal distance from both. Similarly, the first mirror may be arranged to be located between the second mirror and the third mirror, preferably at an equal distance from both. According to some exemplary configurations of the invention, the second light source and the third mirror may be configured to perform rotational oscillation, preferably via controlled motors such as step motors or servo motors.

According to some exemplary configurations of the invention, a light source may be arranged to illuminate essentially all of the target areas from which images are to be taken. In such a configuration, it may not be necessary to use an additional light source and a motor, such as a step motor or a servo motor, to rotate it, as mentioned with the first light source above. According to some exemplary configurations of the invention, in cases where the object for surface defect detection is produced by rotating (e.g., as in a circular knitting or weaving machine), the light source may be connected to rotate along with the rotating component.

According to some exemplary configurations of the invention, the image capturing device can be a camera capable of capturing photo image or video image. According to some exemplary configurations of the invention, the rotational oscillation speed of the image capturing device, the rotational oscillation speed of the first mirror, the rotational oscillation speed of the light source, and the rotational oscillation speed of the second mirror can be set to a predetermined fixed value or can be adjustable to a desired value.

According to some exemplary configurations of the invention, the rotational oscillation of the image capturing device and the rotational oscillation of the light source may be configured such that the area illuminated by the light source on the object at least partially overlaps with the target area captured by the image capturing device. In a preferred configuration of the invention, the area illuminated by the light source essentially overlaps with the target area captured by the image capturing device.

It is a common practice in art that multiple cameras arranged at specific intervals are usually used for quality control in flat weaves. According to the invention, it is possible to photograph the fabric across its entire width using a single rotating camera system. This solution can also be used in quality control machines for flat-flowing fabrics, over weaving looms, and in the quality control of plastics and metals.

According to some exemplary configurations of the invention, the “light” coming from the light source within the scope of this description can be of various wavelengths. The light can be, for example, visible light, infrared light, or ultraviolet light.

According to some exemplary configurations of the invention, the image capturing unit can include a lens that can dynamically change its focal length to a desired level, such as a motorized lens or flexible lenses that change focus using an electromechanical compression method or a liquid lens that operates on the “electrowetting” principle, where the interface between two liquids is modified when voltage is applied. Thus, it becomes possible to obtain a clearer image by focusing before each image capture operation as the image capturing device is rotated (or the mirror is rotated, when used).

### BRIEF DESCRIPTION OF THE FIGURES

FIG. 1: An elevated perspective view of the defect detection system suitable for the invention along with the rotating component of a tubular-shaped object is given.

FIG. 2: An elevated perspective view from another angle of the defect detection system suitable for the invention along with the rotating component of a tubular-shaped object is given.

FIG. 3: A representative perspective view of the defect detection system suitable for the invention, showing the image capturing device, the first light source, and the relevant mirrors.

FIG. 4: Another representative perspective view of the defect detection system suitable for the invention, showing the image capturing device, the first light source, and the relevant mirrors from a different angle.

FIG. 5: A representative side view of the defect detection system suitable for the invention, showing the image capturing device, the first light source, the second light source, and the relevant mirrors.

FIG. 6: A representative side view of the defect detection system suitable for the invention, showing the linear movement of the image capturing device.

FIG. 7: A representative side view of the defect detection system suitable for the invention, showing the image capturing device and the first light source with coaxial and conical mirrors.

FIG. 8: A representative perspective view of a configuration including the image capturing device and the first light source of the defect detection system suitable for the invention.

FIG. 9: A view of the sequential images taken from the tubular-shaped object arranged side by side.

FIG. 10: A view corresponding to the step of correcting the images in FIG. 9.

FIG. 11: A view of the image data fragments obtained from the corrected images.

FIG. 12: A view corresponding to an initial image capturing moment of the image capturing device.

FIG. 13: A view corresponding to a second image capturing moment of the image capturing device.

FIG. 14: A view corresponding to a third image capturing moment of the image capturing device.

FIG. 15: A view of the appearance of a surface defect detection application on a quality control machine for objects in flat flow.

FIG. 16: A view of the appearance of a defect detection application on a bundle as an object consisting of parts arranged side by side in a longitudinal form.

FIG. 17: A view of the appearance of a surface defect inspection application on a weaving machine for a textile object in flat flow.

FIG. 18: A view of an appearance corresponding to the application of light from the back surface of translucent objects.

FIG. 19: A view of the appearance of an application in a weaving machine where the imaging device and the light source are positioned separately.

### DESCRIPTION OF THE COMPONENTS IN THE FIGURES

1. Object
2. Rotating component
3. Separator arms
4. Target area
5. Winding roll
6. Image capturing device
7. First mirror
8. Image sensor
9. First light source
10. First motor
11. Second motor
12. Third motor
14. Second mirror
15. Reflector
16. Reflector sensor
17. First laser source
18. Second laser source
19. Second light source
21. Third mirror
22. Fourth light source
23. Housing
24. Image data piece
25. Image device projection
26. Backlight source

### DETAILED DESCRIPTION OF THE INVENTION

Within the scope of the invention, the term “object” whose surface is to be detected for defects should be understood to correspond to objects characterized by a pattern on a planar surface. For example, textile materials such as woven or knitted fabrics, surfaces of solid objects like plastic, glass, or metal, and a bundle consisting of multiple threads arranged side by side should be considered as “objects” within the scope of the invention. The “object” within the scope of the invention can be in a rotatable state, being connected to a rotating mechanism, or it can be in a stationary state. Additionally, the “object” can also be in a flat-flow state.

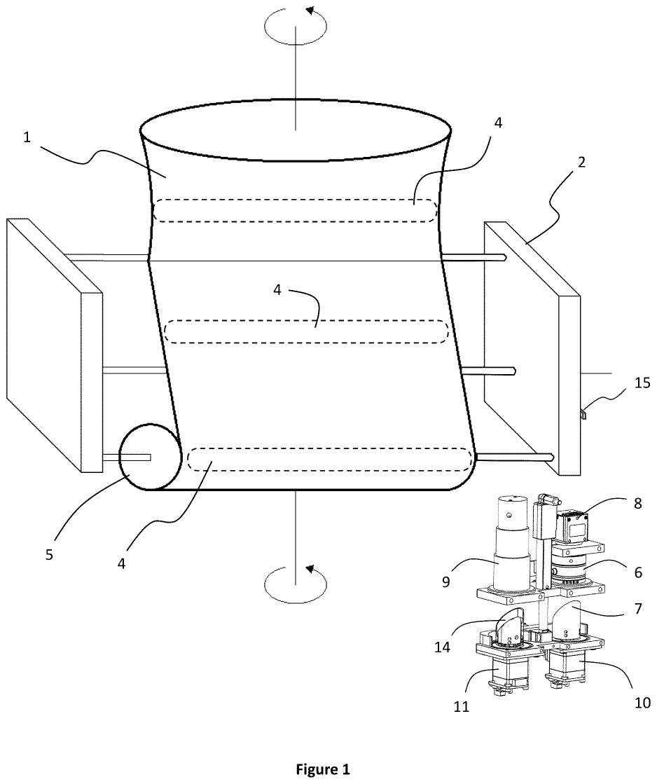

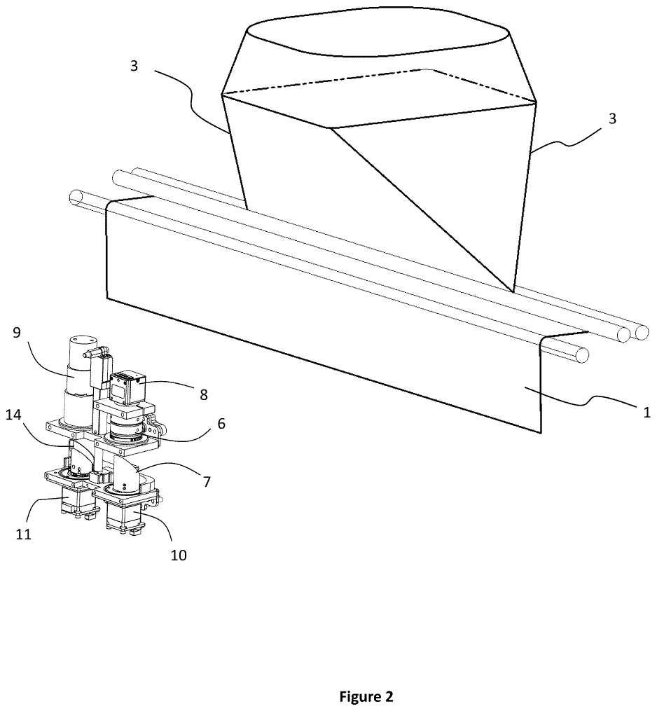

FIG. 1 shows a rotating component (2) of a machine where a tubular-shaped object (1) is produced and the defect detection system. An example of a machine producing tubular-shaped objects with a rotating component may be a circular knitting machine. Tubular textile materials produced by knitting are obtained by knitting yarns fed to each of the multiple knitting needles moving vertically in a circular knitting machine. The knitted textile product (fabric) forms a tubular shape immediately below the knitting needles and extends continuously downward.

As the textile product extends downward from immediately below the knitting needles, it takes on a shape similar to an inverted cone because the textile product is more tensioned in the region where the needles are located. When the textile product reaches the region where the separator arms (3) are located, it takes on a shape similar to an inverted triangular pyramid. The textile product passing through the lower edge of the inverted triangular pyramid takes on a plate/sheet form and is then wound onto a winding roll (5).

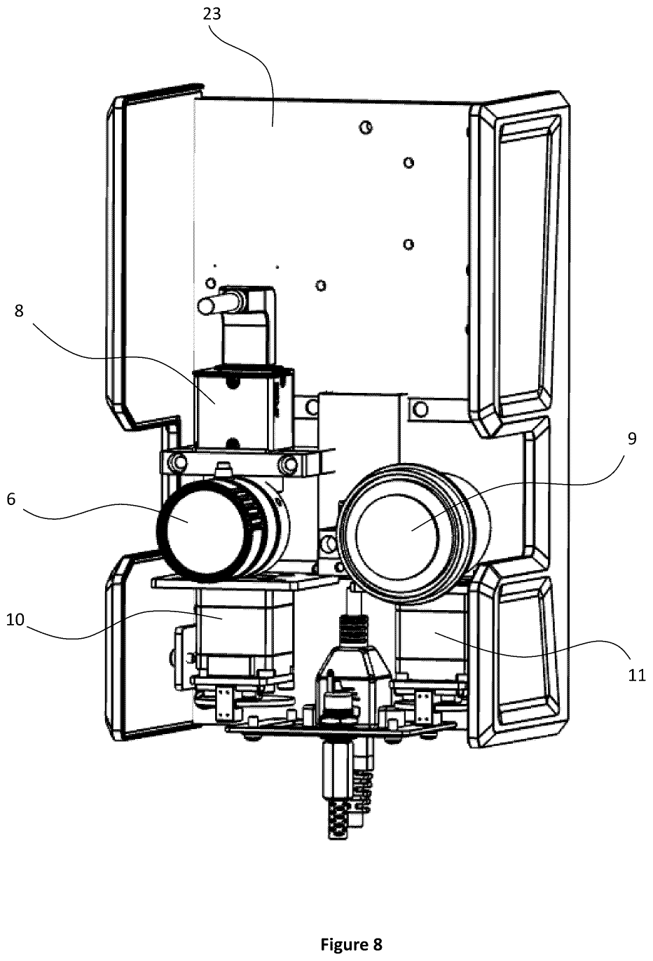

In an embodiment of the invention, an image capturing device (6), such as a camera configured to capture photo or video images, is arranged at a distance from the rotating component (2). In such a configuration, the image capturing device (6) is arranged independently of the rotating component (2). The viewing projection of the image capturing device (6) forms a target area (4) on the textile product for the images to be taken. In an embodiment of the invention, the image capturing device (6) may be arranged within a housing (23). As shown in FIG. 8, the image capturing device (6) is rotated to perform a rotational oscillation by a first motor (10), which is preferably also arranged within the housing (23). The first motor (10) can be connected to the image capturing device (6) from the bottom or the top. Within the scope of the invention, the term “rotational oscillation” should be understood to mean rotating the image capturing device (6) in the clockwise and counterclockwise directions within an angle range of 0° to 180°. The axis of rotation of the image capturing device (6) can preferably be aligned with the axis of rotation of the rotating component (2) and preferably this direction can be perpendicular, parallel or at any angle to the ground plane.

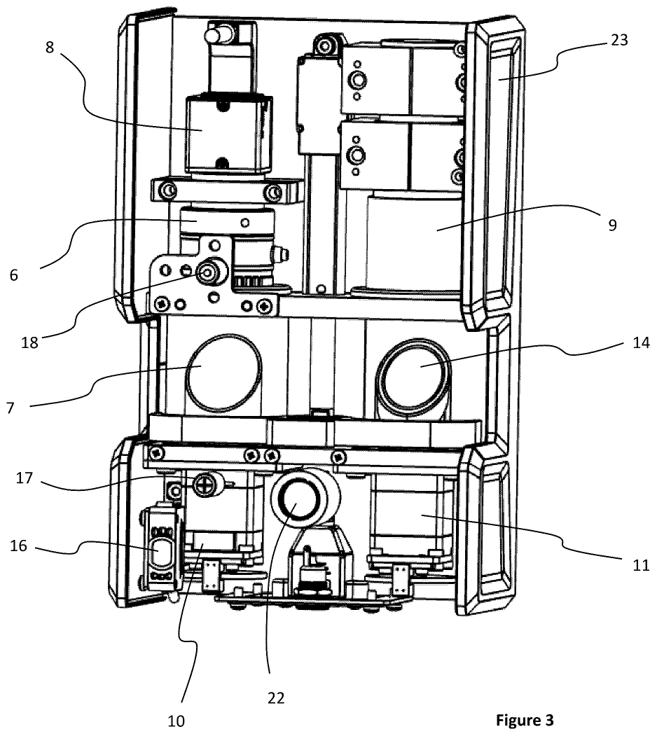

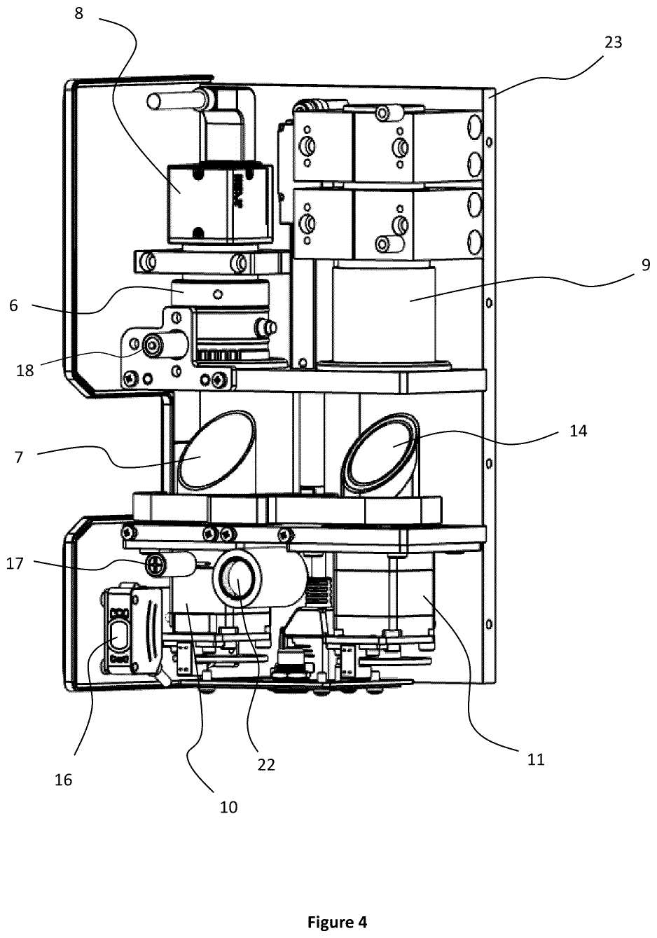

As seen in FIG. 3 or FIG. 4, the image capturing device (6) may also be non-rotatably coupled to the housing (23). In this case, a first mirror (7) can be rotated by the first motor (10) to perform rotational oscillation. The first mirror (7) can have an inclined surface with respect to the vertical (or horizontal) direction, and the inclination angle is preferably 45 degrees. According to this configuration, the images of the target area (4) taken from the tubular object (1) are reflected by the first mirror and transmitted to the image capturing device (6). Again, in this configuration, the first mirror (7) may also be arranged within the housing (23).

According to some exemplary configurations of the invention, the term “motor” should be understood to include both digitally controlled and non-digitally controlled motors. According to some exemplary configurations of the invention, the term “motor” within this description preferably corresponds to digitally controlled motors such as step motors or servo motors. Thus, parameters such as the angle range and speed of the motor's rotational oscillation can be controlled. For instance, the target areas (4) from which images are to be taken for detecting surface defects of a tubular textile product can be taken from the inverted cone-like surface immediately below the knitting needles as mentioned above, or from the inverted triangular pyramid-like surface in the region where the separator arms (3) are located, or from the surface where the textile product takes on a sheet form. In this case, since the width of the textile product can vary, the angle range of the rotational oscillation of the image capturing device (6) (or the first mirror (7)) can also vary.

According to some exemplary embodiments of the invention, the width value of the textile product can be defined by the user as input data to a processor, as explained below, thereby calculating the rotational oscillation angle range. Similarly, this angle range calculation can also be performed for the first light source (or second mirror) and the second light source (or third mirror), as explained below.

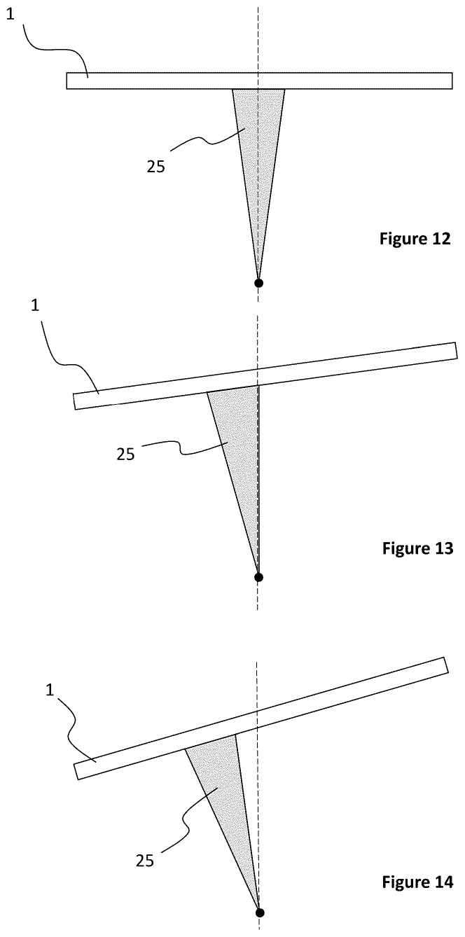

Similarly, by controlling the speed of the “motor,” more precise images can be obtained. This situation can be particularly important due to the rotation of the textile product by the rotating component. As the textile product rotates, it is likely that the images taken when the projection plane of the image capturing device (6) is essentially in the same plane as the target area will have higher clarity (quality). In this case, the rotational speed (or the amount of rotational angle) of the image capturing device (6) (or the first mirror (7)) can be adjusted considering the rotational speed (or the amount of rotational angle) of the textile product. Thus, it becomes possible to position the image capturing device (6) (or the first mirror (7)) in the most optimal position while performing rotational oscillation, meaning positioning the projection plane of the image capturing device (6) (or the projection plane of the first mirror (7)) in essentially the same plane as the target area (4). In a representative case where the object (1) is rotated three times consecutively, the projection of the image capturing device (6) is shown in FIGS. 12 to 14. Here, the term “essentially” should be understood to mean that the angle between the projection plane and the target area plane may be between −5° and +5°. However, in the case of flat objects, this tolerance angle is likely to be greater, for example, an angle range between −10° to +10°.

The angular positions to which the image capturing device (6) (or the first mirror (7)) will be brought during rotational oscillation can be predetermined or calculated just before each image capture operation, in various ways. According to some exemplary embodiments of the invention, a reflector (15) can be coupled to the rotating component (2). A reflector sensor (16), preferably arranged within the housing (23), is configured to detect when it is aligned with the reflector (15). Since the circumferential speed of the rotating component (4) is known, the time interval between the first detection and the second detection of the reflector (15) by the reflector sensor (16) can be fed into the processor to calculate the circumference of the diameter formed by the rotating textile product. This circumference value can be divided by a predetermined fixed number, such as 2048 or 4096, to determine the amount of angle the first motor (10) will rotate each time. Additionally, in cases where a rotary encoder can be connected, angular velocity and position information can be obtained.

In the case of flat objects, for instance, where fabric speed is necessary, speed and position information can be obtained from encoders attached to the rollers through which the fabric passes.

The drive signal frequency (sequence) sent to the first motor (10) can be varied based on the angular position of the image capturing device (6) (or the first mirror (7)). For instance, in an application where the first motor (10) (and consequently the image capturing device (6) or the first mirror (7)) performs a rotational oscillation of 50.7740°, if 25.3370° (half of) 50.7740° is taken as the reference point, and the angles in the clockwise direction are considered as “−” and the angles in the counterclockwise direction are considered as “+”, drive signals can be sent to the first motor (10) at various angles as shown in the following table:

TABLE 1

| first motor angle (°) | first motor angle (°) |
|---|---|
| −0.054 | −0.777 |
| −0.161 | −0.537 |
| −0.322 | −0.322 |
| −21.020 | −0.161 |
| −21.181 | −0.054 |
| −21.289 | 0.054 |
| −25.334 | 0.161 |
| −25.226 | 0.322 |
| −25.065 | 0.537 |
| −24.850 | 0.777 |
| −24.614 | 1.018 |
| −24.378 | 1.258 |
| −20.596 | 4.142 |
| −20.381 | 4.382 |
| −20.220 | 4.597 |
| −20.113 | 4.758 |
| −20.005 | 4.865 |
| −19.898 | 4.973 |
| −19.737 | 5.080 |
| −19.522 | 5.241 |
| −19.282 | 5.456 |
| −19.042 | 5.699 |
| −15.444 | 5.941 |
| −15.229 | 6.184 |
| −15.068 | 9.095 |
| −14.960 | 9.338 |
| −14.853 | 9.552 |
| −14.745 | 9.714 |
| −14.584 | 9.821 |
| −14.369 | 9.928 |
| −14.137 | 10.036 |
| −13.904 | 10.197 |
| −10.412 | 10.412 |
| −10.197 | 10.645 |
| −10.036 | 10.877 |
| −9.928 | 14.369 |
| −9.821 | 14.584 |
| −9.714 | 14.745 |
| −9.552 | 14.853 |
| −9.338 | 14.960 |
| −9.095 | 15.068 |
| −8.852 | 15.229 |
| −8.610 | 15.444 |
| −5.699 | 15.684 |
| −5.456 | 15.923 |
| −5.241 | 16.163 |
| −5.080 | 19.282 |
| −4.973 | 19.522 |
| −4.865 | 19.737 |
| −4.758 | 19.898 |
| −4.597 | 20.005 |
| −4.382 | 20.113 |
| −4.142 | 20.220 |
| −3.901 | 20.381 |
| −3.661 | 20.596 |
| | 20.833 |
| | 21.069 |
| | 24.850 |
| | 25.065 |
| | 25.226 |
| | 25.334 |

According to some exemplary arrangements of the invention, it may not be necessary to specifically illuminate the target areas (4) where images will be taken on the object (1). For example, the light intensity in the environment where the imaging device (6) operates may be sufficient on its own. Therefore, the use of light sources directed at the target areas (4), which will be detailed below, may be optional.

According to an embodiment of the invention, as seen in FIG. 8, a first light source (9) can be arranged on one side of the image capturing device (6) and preferably within the housing (23). The light beam from the first light source (9) is configured to illuminate the target area (4) on the textile product. The first light source (9) is rotated to perform rotational oscillation by a second motor (11), which is also preferably arranged within the housing (23). The second motor (11) can be coupled to the first light source (9) from the bottom or the top. Like the first motor (10), the second motor (11) can perform rotational oscillation within an angle range of 0° to 180°. The axis of rotation of the first light source (9) is aligned with the axis of rotation of the rotating component (2), and this direction is preferably perpendicular to the ground plane.

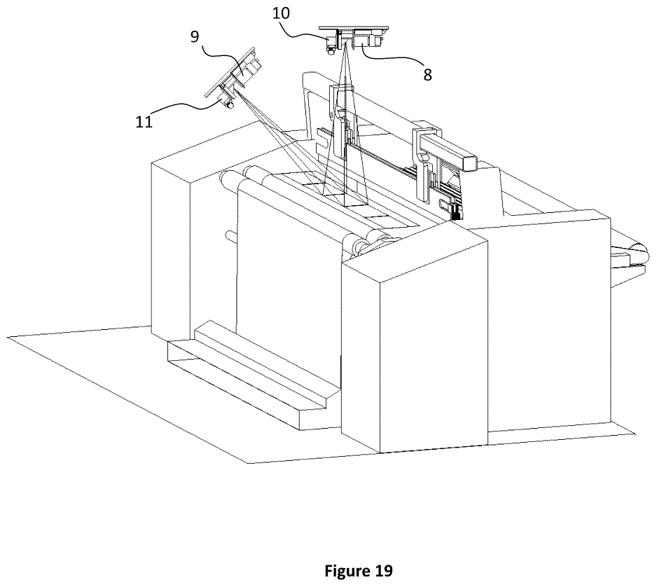

According to some exemplary embodiments of the invention, as shown in FIG. 19, the image capturing device (6) (and other components associated with it, such as the first mirror, image sensor, first motor) and the first light source (9) (and other components associated with it, such as the second motor, second mirror) can be configured to be in separate locations from each other. Similarly, in the case of using multiple light sources, these other light sources can also be configured to be in separate locations from each other.

As seen in FIG. 3 or FIG. 4, the first light source (9) can also be non-rotatably coupled to the housing (23). In this case, a second mirror (14) can be rotated by the second motor (11) to perform rotational oscillation. The second mirror (14) has an inclined surface with respect to the vertical (or horizontal) direction, and the inclination angle is preferably 45 degrees. According to this configuration, the light beam sent from the first light source (9) is reflected by the second mirror (14) and directed to the target area (4) on the tubular object (1). Again, in this configuration, the second mirror (14) can also be arranged within the housing (23).

Similar to the first motor (10), the speed of the second motor (11) may also be controlled to obtain more precise images. The light beam projection plane may be sent to be essentially in the same plane as the target area plane. In this case, the rotational speed (or the amount of rotational angle) of the first light source (9) (or the second mirror (14)) may be adjusted considering the rotational speed (or the amount of rotational angle) of the textile product. Thus, it becomes possible to position the first light source (9) (or the second mirror (14)) in the most optimal position while performing rotational oscillation, meaning positioning the projection plane of the first light source (9) (or the projection plane of the second mirror (14)) in essentially the same plane as the target area (4). Here again, the term “essentially” should be understood to mean that the angle between the projection plane and the target area plane may be between −5° and +5°. However, in the case of flat objects, this tolerance angle is likely to be greater, for example, an angle range between −10° to +10°.

The angular positions to which the first light source (9) (or the second mirror (14)) will be brought during rotational oscillation may be predetermined or calculated just before each image capture operation, in various ways. For this purpose, the reflector (15) and reflector sensor (16) arrangement mentioned above may be used. This allows the calculation of the angular amount that the second motor (11) will rotate each time.

The drive signal frequency (sequence) sent to the second motor (11) may be varied based on the angular position of the first light source (9) (or the second mirror (14)). According to the previous example, in an application where the second motor (11) (and consequently the first light source (9) or the second mirror (14)) performs a rotational oscillation of 50.7740°, drive signals can be sent to the second motor (11) at various angles as shown in the following table:

TABLE 2

| Second motor angle (°) | Second motor angle (°) |
|---|---|
| −0.054 | 4.460 |
| −0.161 | 4.728 |
| −0.261 | 4.943 |
| −13.434 | 5.104 |
| −13.534 | 5.211 |
| −13.641 | 5.265 |
| −19.837 | 5.265 |
| −19.837 | 5.319 |
| −19.783 | 5.426 |
| −19.676 | 5.587 |
| −19.515 | 5.802 |
| −19.300 | 6.071 |
| −15.122 | 9.339 |
| −14.907 | 9.607 |
| −14.746 | 9.822 |
| −14.638 | 9.983 |
| −14.585 | 10.091 |
| −14.585 | 10.144 |
| −14.531 | 10.144 |
| −14.423 | 10.198 |
| −14.262 | 10.306 |
| −14.047 | 10.467 |
| −10.032 | 10.682 |
| −9.818 | 10.950 |
| −9.656 | 14.257 |
| −9.549 | 14.526 |
| −9.495 | 14.741 |
| −9.495 | 14.902 |
| −9.442 | 15.009 |
| −9.334 | 15.063 |
| −9.173 | 15.063 |
| −8.958 | 15.117 |
| −5.055 | 15.224 |
| −4.840 | 15.385 |
| −4.679 | 15.600 |
| −4.572 | 19.522 |
| −4.518 | 19.737 |
| −4.518 | 19.898 |
| −4.464 | 20.006 |
| −4.357 | 20.059 |
| −4.196 | 20.059 |
| −3.981 | 20.113 |
| −3.712 | 20.220 |
| −0.416 | 20.382 |
| −0.148 | 20.596 |
| 0.067 | 20.865 |
| 0.228 | 24.372 |
| 0.336 | 24.640 |
| 0.390 | 24.855 |
| 0.390 | 25.016 |
| 0.443 | 25.124 |
| 0.551 | 25.178 |
| 0.712 | 25.178 |
| 0.927 | 25.231 |
| 1.195 | 25.339 |
| | 25.500 |
| | 25.715 |
| | 29.935 |
| | 30.150 |
| | 30.311 |
| | 30.418 |

According to some exemplary embodiments of the invention, drive signals may be sent simultaneously to the first motor (10) and the second motor (11). The angular values shown in Table 1 and Table 2 correspond to a configuration where drive signals are sent simultaneously to these two motors. When there is a distance between the image capturing device (6) (or the first mirror (7)) and the first light source (9) (or the second mirror (14)), the angular positions of the first motor (10) and the second motor (11) are often not the same, as shown in Table 1 and Table 2. This is necessary to ensure that the projection area of the image capturing device (6) (or the first mirror (7)) essentially overlaps with the area illuminated by the first light source (9) (or the second mirror (14)).

According to some exemplary embodiments of the invention, drive signals may be sent to the first motor (10) and the second motor (11) at different times.

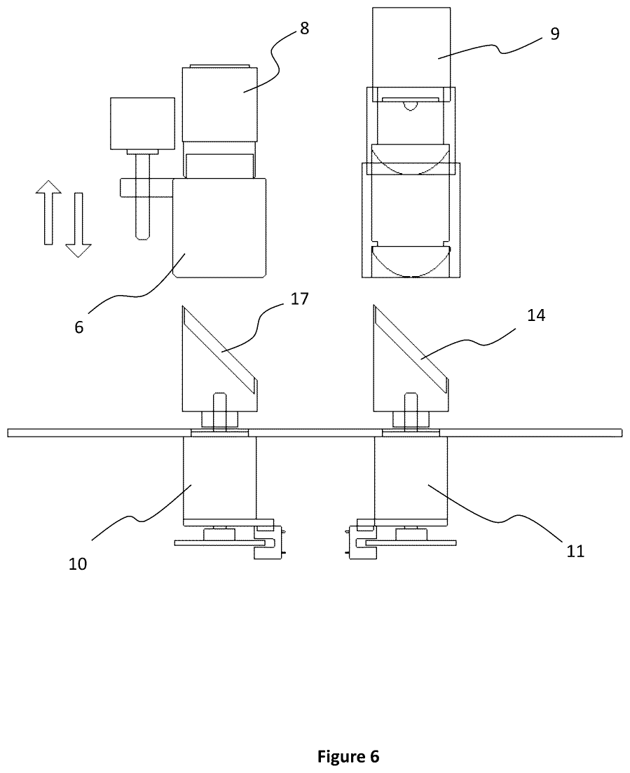

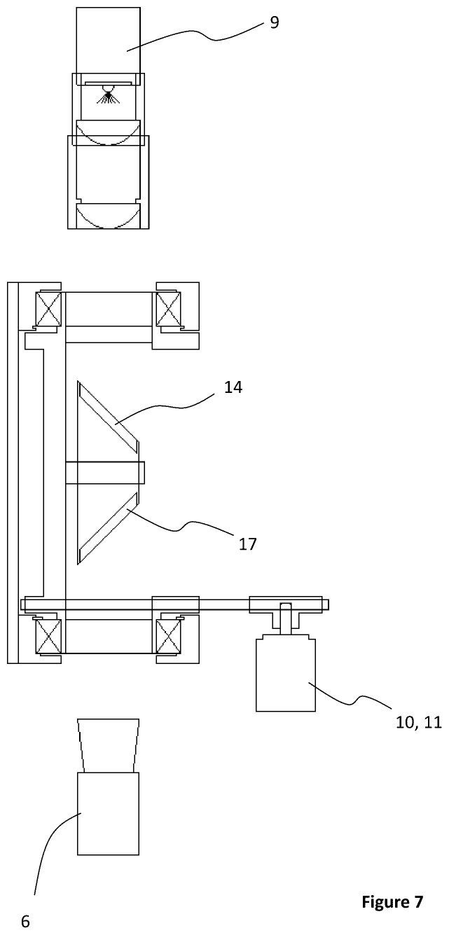

According to some exemplary embodiments of the invention, the first light source (9) (or the second mirror (14)) may be positioned at any location relative to the image capturing device (6) (or the first mirror (7)). For example, it is possible to configure the first light source (9) to be on the same axis as the image capturing device (6). In this case, the first light source (9) and the image capturing device (6) may be rotated by the same motor. In an alternative exemplary configuration shown in FIG. 7, the first light source (9) may be arranged at the top, and the image capturing device (6) may be arranged at the bottom. In this configuration, the first mirror (7) and the second mirror (14) may be positioned to define a conical geometry. In this case, the mirrors (7, 14) may be made to perform rotational oscillation by a single motor.

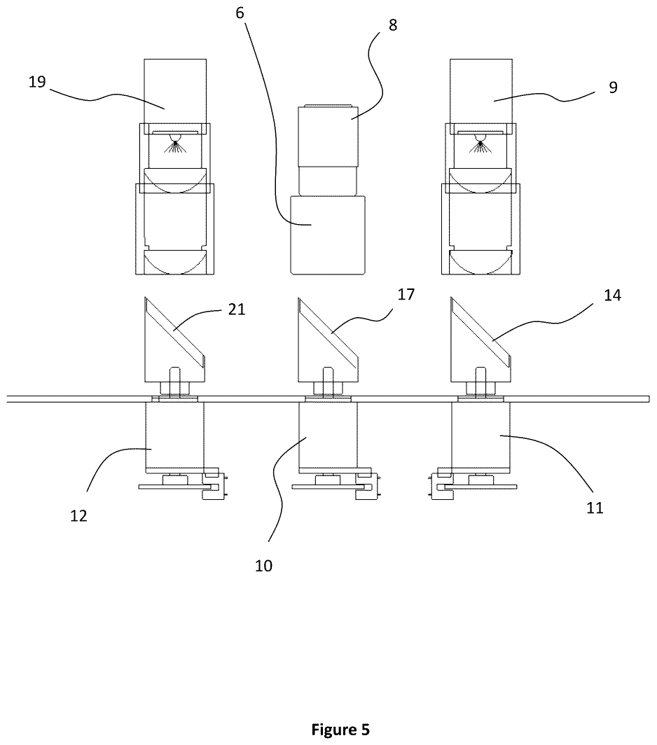

According to some exemplary embodiments of the invention, as shown in FIG. 6, the image capturing device (6) (or the first mirror (7)) may be moved linearly, for example, vertically or horizontally, in addition to rotational oscillation. This allows the size of the target area (4) from which images are taken to be adjusted. Similarly, the light source (or additional light sources as explained below) may be moved linearly, for example, vertically or horizontally, in addition to rotational oscillation. Similarly, in a configuration where the image capturing device and/or the light source is fixed, the corresponding mirrors (7, 14) may also be moved linearly in addition to rotational oscillation.

According to some exemplary embodiments of the invention, in addition to the first light source (9), a second light source (19) driven by a third motor (12) may be arranged on the other side of the image capturing device (6) and preferably within the housing (23). In this case, according to some exemplary embodiments of the invention, the distance between the second light source (19) and the first light source (9) to the image capturing device (6) may be the same, and the arrangement may be made such that the image capturing device (6) is in the middle, with the second light source (19) and the first light source (9) positioned opposite each other. Similar to the first light source (9), a configuration where the second light source (19) is fixed may also be possible. In this case, a third mirror (21) may be rotated by the third motor (12) to perform rotational oscillation. In such a configuration, the light beam from the second light source (19) also illuminates the same target area as the light beam from the first light source (9). The operating principle of the second light source (19) is essentially the same as that of the first light source (9). Similarly, the operating principle of the third mirror (21) is essentially the same as that of the second mirror (14).

According to some exemplary configurations of the invention, while the image capturing device (6) is rotated by the first motor, the first light source (9) may be kept stationary, and the second mirror (14) can then be rotated. Alternatively, the first light source (9) may be rotated while the image capturing device (6) can be kept stationary, and the first mirror (7) can be rotated, creating various alternative combinations. Similarly, alternative combinations may be made in a configuration where the second light source (19) is used.

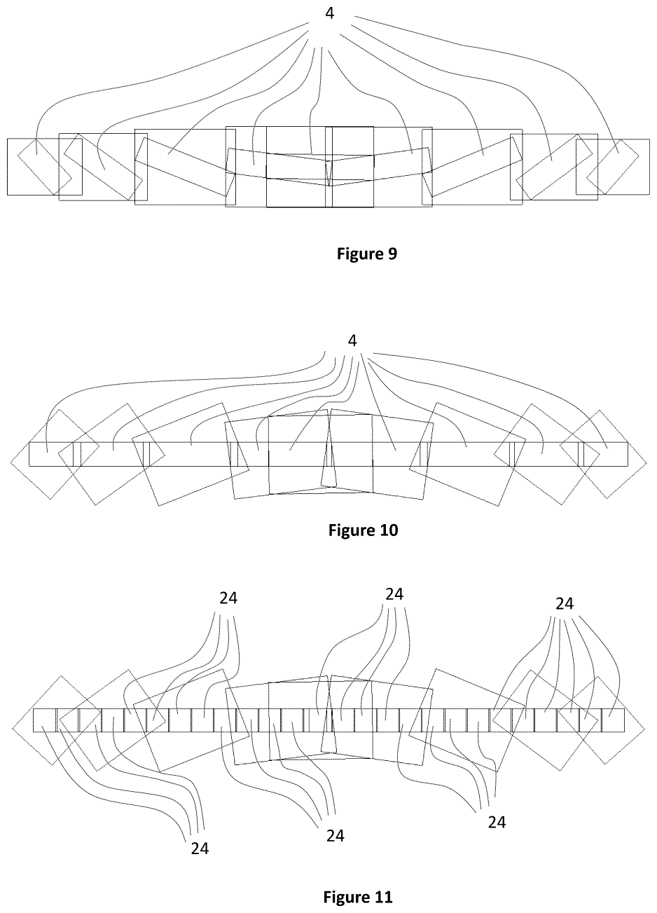

In each complete rotation of the tubular object (1), the image capturing device (6) can capture multiple images, for example, 18, 20, 22, 24, etc. FIG. 9 shows an exemplary configuration where 9 images are captured (18 images in a full rotation) while the tubular object (1) is rotated halfway and brought into a sheet form. In this example, the image capturing device (6) is stationary, and the first mirror (7) performs rotational oscillation. From left to right, the tilt of the images taken from the tubular object (1) decreases with respect to the horizontal direction, and when the images are taken from the exact center of the tubular object (1), they become parallel to the horizontal. Subsequently, as the images are taken from the exact center of the tubular object (1) to the right, the tilt of the images with respect to the horizontal direction increases.

The image capturing device (6) is communicated with an image sensor (8) such as a CCD sensor, CMOS sensor, or photomultiplier sensor, and the images captured by the image capturing device (6) are converted into data by the image sensor (8) for further processing in the processor. The image capturing device (6) may be equipped with a dynamically focusing lens as known in the art, such as a liquid lens or a motorized lens. This allows the focal distance of the image to be automatically adjusted before each image capture operation. The images captured, as shown in FIG. 9, are transmitted to the processor interfaced with the image sensor (8) and then corrected as shown in FIG. 10. This image correction process may involve several stages. For example, when the captured images from the previous stage are appropriately rotated and placed side by side, the image of the tubular object (1) can be transformed into a data set that extends horizontally in an orderly manner. The overlapping parts of consecutive images can be considered both when processing the first image data and the subsequent second image data. This allows for a more reliable detection of any surface defects that may remain in the overlapping area. The amount of overlap in consecutive images can be calculated as a function of parameters such as the width of the tubular object (1), its rotation speed, the speed of the image capturing device (6) (or the first mirror (7)), and the image capture sequence.

As shown in FIG. 11, the image data can be divided into small image data fragments (24) arranged side by side (26 image data pieces are shown in the example in FIG. 11), and these image data fragments (24) can be used to perform defect detection analysis on the surface of the tubular object (1). The processor is equipped with an algorithm to perform the image data analysis. This algorithm can be a dedicated software or application, a pre-trained machine learning software, or a machine learning software that self-trains from images taken during production, or it can be an image processing software known in the art. As a result of the analysis of the captured image data, if a defect is detected, various control actions such as stopping the machine producing the tubular object, generating audible or visual alerts, etc., can be taken. For these control actions, the processor can be interfaced with the control unit of the machine producing the tubular object.

Depending on the material properties, texture, and pattern of the tubular object (1), light of a certain wavelength can be directed onto the object for a specific period, followed by light of a different wavelength for another period. For example, visible light can be emitted from the first light source (9) (or the second light source (19)) for a certain period, and ultraviolet light can be emitted from a fourth light source (22), preferably arranged within the housing (23), for another period. While light is emitted from the fourth light source (22), the emission from other light sources can be stopped, but the image capturing process can continue. Additional light sources can also be used in addition to this fourth light source (22).

In a preferred configuration of the invention, the distance of the tubular object (1) to the image capturing device (6) can be determined before the image capturing process. This distance information can be used to set the initial value of the focus distance of the image capturing device (6). For this purpose, a first laser source (17) and/or a second laser source (18), preferably arranged within the housing (23), can be used as shown in FIG. 3 or FIG. 4. According to some exemplary embodiments of the invention, the first laser source (17) can be positioned at a vertical distance from the second laser source (18). The average of the distance information obtained from these two laser sources (17, 18) can be taken.

The first laser source (17) or the second laser source (18) can also be used to control the orientation of the tubular object (1). For example, using a cross laser source, the orientation of the object can be controlled based on the laser mark falling on the tubular object.

Although an exemplary system for detecting surface defects of a textile product produced on a circular knitting machine has been described above, it should be understood that the system suitable for the invention can be adapted to circular weaving machines, flat knitting, or flat weaving machines. For example, when the system suitable for the invention is used to detect defects in a textile product produced on a flat knitting or flat weaving machine, it becomes possible to detect surface defects in a form similar to the sheet form of the textile material before it is wound onto the winding roll (5) as mentioned in the example application above.

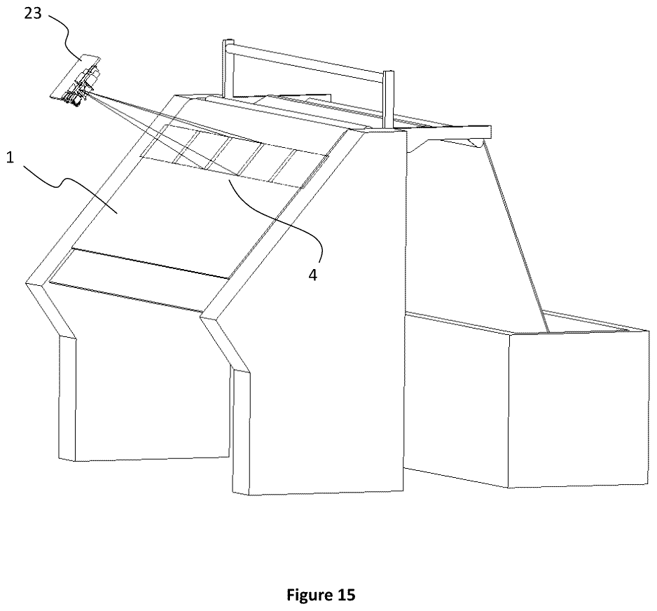

As mentioned earlier, it is possible to apply the system according to the invention to objects that are in a flat-flow state. For example, as shown in FIG. 15, it is possible to detect surface defects on an object in flat-flow on a quality control machine. This quality control machine can be used not only for textile products but also for the surface defect detection of objects in flat sheet form. When it is a textile product, defects in the weft and/or warp threads of its fabric can be detected.

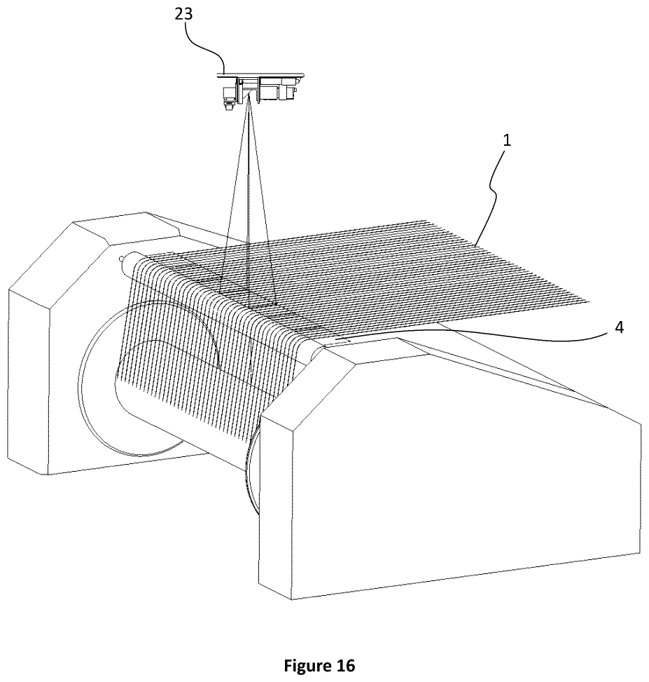

FIG. 16 shows an application in which the object being detected for defects is a bundle consisting of parts arranged side by side in a longitudinal form. Such an object can, for example, be an object consisting of threads arranged side by side. In such a case, defects in the object formed by this bundle of threads can be detected using an arrangement according to the invention while the object is in a rebeaming machine.

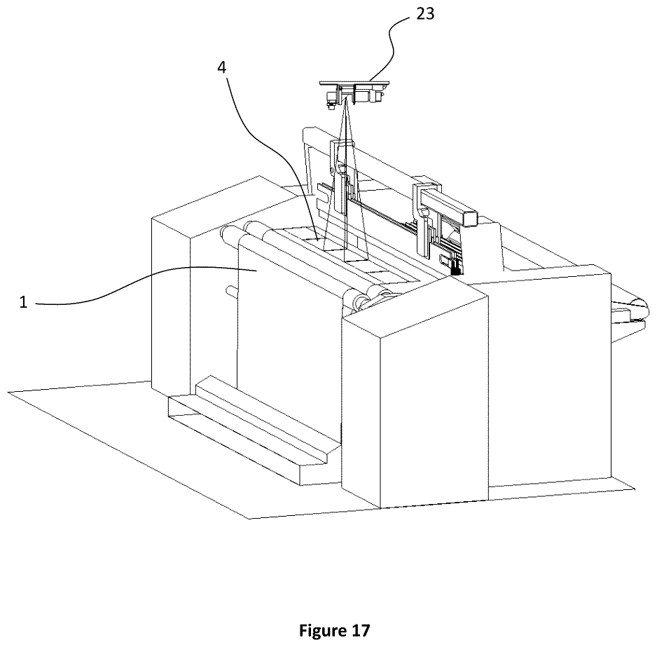

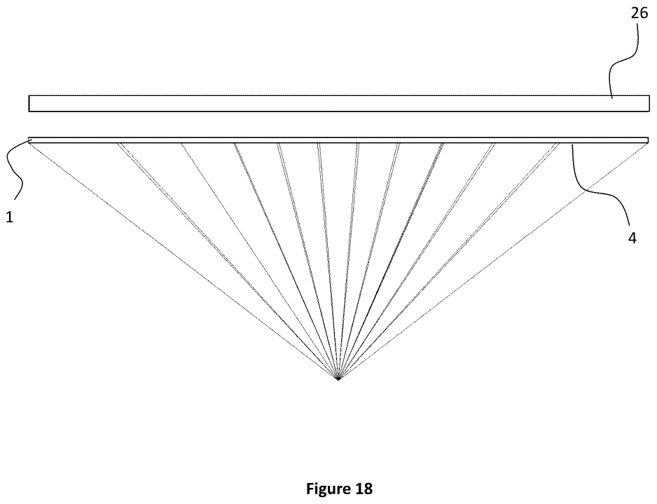

FIG. 17 shows the detection application according to the present invention when the object to be detected for defects is specifically a woven textile product, with the detection performed while the object is in a weaving machine.

When the object to be detected for surface defects is made of a translucent material, a backlight source (26) configured to illuminate the back side of the object can be used. The activation/deactivation of the backlight source (26) can preferably be controlled digitally. The backlight source (26) can emit light together with the other light sources (first light source (9), second light source (19)) or independently of the other light sources, or the light can be cut off. The area illuminated by the backlight source (26) corresponds to the same parts on the front and back surfaces of the object (1) as the target area (4) illuminated by the first light source (9) and/or the second light source (19).

1. A system for actively detecting defects on the surface of an object (1) with a surface area, the system comprising: a calibratable image capturing unit configured to capture images from at least one target area (4) of the object (1), the unit comprising an image capturing device (6) and an optional first mirror (7) configured to transmit images from at least one target area (4) to the image capturing device (6);
an image sensor (8) for converting the images obtained from the image capturing unit into data;
a processor communicating with the image sensor (8) for analyzing the data received from the image sensor (8);
at least one light source,
characterized in that at least the image capturing device (6) or the optional first mirror (7) is configured to perform a rotational oscillation.

2. The system according to claim 1, wherein the image capturing device (6) configured to perform rotational oscillation is interfaced with a controlled first motor (10) such as a step motor or servo motor, and the optional first mirror (7) is interfaced with the first motor (10).

3. The system according to claim 2, wherein the at least one light source comprises a first light source (9) interfaced with a controlled second motor (11) such as a step motor or servo motor to perform rotational oscillation for directing light to the said target area (4), and in that the system comprises an optional second mirror (14) configured to perform rotational oscillation and interfaced with the second motor (11) to transmit light from the first light source (9) to the at least one target area (4).

4. The system according to claim 3, wherein the at least one light source comprises a second light source (19) interfaced with a controlled third motor (12) such as a step motor or servo motor to perform rotational oscillation for directing light to the target area (4), and in that the system comprises an optional third mirror (21) configured to perform rotational oscillation and interfaced with the third motor (12) to transmit light from the second light source (19) to the at least one target area (4).

5. The system according to claim 1, further comprising a backlight source (26) configured to illuminate the back side of the object (1).

6. The system according to claim 4, wherein the image capturing device (6) is arranged to be positioned between the first light source (9) and the second light source (19) and at an equal distance therefrom.

7. The system according to claim 1, wherein the at least one light source comprises a light source configured to illuminate essentially the whole of the at least one target area.

8. The system according to claim 3, wherein the rotational oscillation of the image capturing device (6) and the rotational oscillation of the first light source (9) are configured such that the area illuminated by the light from the first light source (9) on the object (1) at least partially overlaps with the target area (4) captured by the image capturing device (6).

9. The system according to claim 1, wherein the image capturing unit (6) comprises a lens capable of dynamically changing its focal length, such as a liquid lens or a motorized lens.

10. The system according to claim 3, further comprising a housing (23) comprising the image capturing device (6), the optional first mirror (7), the first motor (10), the first light source (9), the optional second mirror (14), and the second motor (11).

11. The system according to claim 1, wherein the image capturing device (6) and the optional first mirror (7) are configured such that the projection plane of the device (6) or the mirror (7) is essentially in the same plane as the target area (4).

12. The system according to claim 3, wherein the first light source (9) and the optional second mirror (14) are configured such that the light beam projection plane of the first light source (9) or the second mirror (14) is essentially in the same plane as the target area (4).

13. The system according to claim 12, wherein the image capturing device (6) and the first light source (9) are configured to be on the same axis, and the first mirror (7) and the second mirror (14) are configured to define a conical geometry.

14. The system according to claim 3, wherein the image capturing device (6), or the optional first mirror (7), or the first light source (9), or the second mirror (14) is configured to move linearly.

15. The system according to claim 1, wherein the processor is equipped with an image data analysis algorithm configured to correct the image data received from the image sensor (8) and to divide the image data into data fragments (24).

16. The system according to claim 4, wherein the light emitted from the first light source (9) or the second light source (19) is in the visible light wavelength.

17. The system according to claim 16, further comprises at least one fourth light source (22) capable of emitting light in the ultraviolet wavelength.

18. A system for actively detecting surface defects of a tubular object (1) in a machine with a rotating component (2) for the production of the tubular object (1), the system comprising: a calibratable image capturing unit configured to capture images from at least one target area (4) of the tubular object (1), the unit comprising an image capturing device (6) and an optional first mirror (7) configured to transmit images from at least one target area (4) to the image capturing device (6);
an image sensor (8) for converting the images obtained from the image capturing unit into data;
a processor communicating with the image sensor (8) for analyzing the data received from the image sensor (8);
at least one light source (9, 19),
characterized in that at least the image capturing device (6) or the optional first mirror (7) is configured to perform a rotational oscillation.

19-22. (canceled)

23. A system for actively detecting defects on the surface of an object (1) with a surface area, the system comprising: a calibratable image capturing unit configured to capture images from at least one target area (4) of the object (1), the unit comprising an image capturing device (6) and an optional first mirror (7) configured to transmit images from at least one target area (4) to the image capturing device (6);
an image sensor (8) for converting the images obtained from the image capturing unit into data;
a processor communicating with the image sensor (8) for analyzing the data received from the image sensor (8);
at least one optional light source,
characterized in that at least the image capturing device (6) or the optional first mirror (7) is configured to perform a rotational oscillation.
